(19) United States
(12) Patent Application Publication
(10) Pub. No.: US 2022/0133155 A1
(43) Pub. Date: May 5, 2022

(54) PATIENT MONITORING SYSTEM

(71) Applicants: Bellagunda Renganalthan, Chennai (IN); Sridhar Nagaiyan, chennai (IN)

(72) Inventors: Bellagunda Santhanakrishnan Renganathan, Chennai (IN); Sridhar Nagaiyan, Chennai (IN); Preejith Sreeletha Premkumar, Chennai (IN); Mohanasankar Sivaprakasam, Chennai (IN)

(21) Appl. No.: 17/431,311

(22) PCT Filed: Feb. 11, 2020
(86) PCT No.: PCT/IN2020/050137

§ 371 Date: Aug. 16, 2021

(30) Foreign Application Priority Data
Feb. 14, 2019 (IN) 201941005889

(51) Int. Cl. A61B 5/0205; A61B 5/28; A61B 5/00; A61B 5/01

(52) U.S. Cl. CPC A61B 5/0205; A61B 5/28; A61B 5/742; A61B 5/747; A61B 5/6823; A61B 2562/0271; A61B 5/6832; A61B 5/01; A61B 2560/0462; A61B 2562/0219; A61B 5/6828

(57) ABSTRACT

Embodiments herein disclose a patient monitoring system (100). The patient monitoring system (100) includes an ECG electrode (110), comprises multiple built-in sensors (110e), configured to measure patient information and stream the patient information. Further, a patient monitor (120) is configured to receive the streamed patient information from the ECG electrode (110) and display the streamed patient information. The patient monitoring system (100) is primarily targeted for patients in ICU who are at risk of acquiring pressure ulcers or VAP or Sepsis. A wearable device in the form factor of the ECG electrode with multiple built-in sensors to monitor patient bed position, head up elevation angle, skin temperature, heart rate and respiratory rate. Information from smart ECG electrode is relayed either through a wired or wireless medium and displayed through the standard patient monitor or using a gateway device.

### FIELD OF INVENTION

The present disclosure relates to a wearable device, and more specifically related to an ECG electrode that can act as an interface to pick up ECG signal and as well measure patient position, activity, head up elevation angle, skin temperature, heart rate and respiratory rate. The present application is based on, and claims priority from an Indian Application Number 201941005889 filed on 14 Feb., 2019 and PCT/IN2020/050137 filed on 11 Feb., 2020 the disclosure of which is hereby incorporated by reference herein.

### BACKGROUND OF INVENTION

Hospital-acquired pressure ulcers (HAPUs) (normally known as bedsores) are injuries caused to skin and its underlying tissue resulting from prolonged pressure on a skin of the patient. The HAPUs often develop on the skin covering bony prominences such as the heels, ankles, hip, and tailbone. Blood supply is disrupted in this region under the influence of pressure, shear, friction or a combination of any of these. This has detrimental effects on skin and its underlying tissue which breakdowns due to insufficient blood circulation. The patients who are at risk include subjects with reduced mobility, reduced perception of sensory information and malnourished and dehydrated geriatric patients. Moisture surrounding the skin is also a contributing factor towards the development of the HAPUs.

High and moderate risk patients are provided care to prevent the occurrence of HAPU by following the hospital's turn protocol. The patient's position is changed every two hours to alternating lateral and supine positions by the caregiver and a manual log of the changed position is recorded. In Intensive Care Units (ICUs) and hospital wards, such a turning procedure is not always followed strictly because of low caregiver compliance to turning protocols. Difficulty in continuously monitoring patient position, lack of a system which can provide turn reminders or alerts and suboptimal caregiver staffing ratio increase the occurrence of HAPUs.

Pneumonia is a common lung infection which is characterized primarily by inflammation of the alveoli in the lungs. Ventilator-associated pneumonia (VAP) is a type of pneumonia which commonly occurs in the patients who are on mechanical ventilation for 48 hours or more. The VAP occurs mainly because of the invasion of the microorganisms in the lower respiratory tract and lung parenchyma. The VAP can lead to other complications such as organ failure, breathlessness, and lung abscess.

In order to avoid the complications associated with the VAP, a VAP bundle was introduced. It is a group of evidence-based protocols used to prevent the occurrence of the VAP. The VAP bundle contains five components, the elevation of the head of the bed to 30-45 degrees, daily assessment of sedation vacation and readiness to extubate, deep venous thrombosis prophylaxis, peptic ulcer disease prophylaxis and daily oral care with chlorhexidine. The elevation of the head of the bed is an important component of the bundle and is highly correlated with the reduction of VAP incidence. In a clinical trial considered, the patients in semi-recumbent position had only 8% VAP incidence compared to 34% VAP incidence, when in a supine position.

In spite of introducing VAP bundle for every 1,000 ventilator days, the incidence of VAP ranges from 13 to 51. This is due to the low rate of compliance with the VAP bundle. In a clinical setting, the overall conformity of the bundle stands at 36.5%. More than 90% of nurses believe that lack of rigid monitoring of VAP care bundle is the main reason for low adherence to VAP care bundle. Keeping the head up angle at 30-45 degrees is a challenging task and the main difficulty lay in continuously monitoring the bed elevation angle. The problems in continuous monitoring of patient head up angle, lack of a system that can provide reminders or alerts, and suboptimal caregiver staffing ratio increase the occurrence of VAP. The existing system discloses about the continuous measurement of head up angle for VAP bundle compliance.

The existing multimodal monitors despite being connected to many electrodes on the patient's body are not able to measure head up angle, an important component of the VAP bundle to reduce ventilator-associated pneumonia or monitor patient position to reduce pressure sores and alert caregivers when it is time to turn a patient and change their position or detect patient falls or accidental bed exits.

The existing system doesn't disclose about comparing the patient orientation information with a preset turn threshold to determine whether the patient has been turned. Further, the existing system doesn't disclose about determining that the patient orientation information has exceeded the preset turn thresholds i.e., the patient has not been turned, updating the orientation information of the patient by the wearable device to a cloud system central server through the gateway in real time. Further, the existing system doesn't disclose about assisting clinicians in the conduct of passive leg raising test or Passive leg raise (PLR) test accurately an important manoeuvre in critical care units.

Thus, it is desired to address the above-mentioned disadvantages or other shortcomings or at least provide a useful alternative.

### OBJECT OF INVENTION

The principal object of the embodiments herein is to provide a patient monitoring system.

### SUMMARY OF INVENTION

Accordingly, the embodiments herein provide patient monitoring system. The patient monitoring system includes an ECG electrode (i.e., smart ECG electrode) coupled with multiple built-in sensors. The proposed ECG electrode is configured to measure patient information and stream the patient information. A patient monitor configured to receive the streamed patient information, and display the streamed patient information.

In an embodiment, the streamed patient information is displayed through at least one of a gateway device and a cloud connected system.

In an embodiment, the patient monitor alerts a caretaker at regular intervals based on at least one of a configurable hospital turn protocol and a configurable ventilator-associated pneumonia (VAP) bundle protocol.

In an embodiment, the patient monitor and/or the gateway device alerts a caretaker at regular intervals through a message, an alarm or push notification.

In an embodiment, the patient monitor and/or the gateway device alerts a caretaker at regular intervals based a predefined value.

In an embodiment, the smart ECG electrode acts as a master node is connected to a set of passive ECG electrodes through a wired setup.

In an embodiment, the patient information is at least one of a patient bed position, patient head up elevation angle, patient skin temperature, patient respiratory rate, patient heart rate, patient bed exit, arrhythmia, ischemic episodes, and Cardio-respiratory arrests.

In an embodiment, the ECG electrode is placed on the chest of the patient, and wherein the smart ECG electrode is placed on a lower limb of the patient to measure the leg raise angle of the patient to allow the caretaker to carry out the patient leg raising test.

In an embodiment, the smart ECG electrode is placed on the chest of the patient, and wherein at least one built-in sensor is placed on the lower limb of the patient to measure of head up angle of the patient and the leg raise angle of the patient to allow a caretaker to carry out the passive leg raising test.

In an embodiment, the ECG electrode comprises a computation circuit, an ECG acquisition analog front end (AFE) circuit, a power management circuit, a transmission circuit, and at least one of a temperature sensor, an accelerometer, a magnetometer, and a gyroscope.

In an embodiment, the computation circuit, the ECG acquisition AFE circuit, the power management circuit, the transmission circuit, and at least one of the temperature sensor, the accelerometer, the magnetometer, and the gyroscope are surrounded by a metal lead.

In an embodiment, the patient monitor is connected with an electronic device (e.g., smart watch, smart band or the like). In an example, the smart watch receives the patient information from the smart ECG electrode.

In an embodiment, the smart ECG electrode includes various sensors and processing unit that will be housed in a Printed Circuit Board (PCB) which is of circular shape with a hole in a center of approximately the size of an ECG lead. The smart ECG electrode has a physical structure and appearance of a normal clinical grade ECG electrode.

In an embodiment, the PCB with the center hole will be attached to ECG electrode through the ECG lead and adhesive. Among various sensors, a temperature sensor will be attached on a backside of the PCB and closer to the skin to accurately measure the skin temperature. Possibly a punched hole of size of the temperature sensor might be present on the ECG electrode to place the sensor in close proximity with the skin to accurately detect the skin temperature. A single to multiple electrical conductive surface will be present along the various regions of the PCB, connected to the specially designed enclosures based on various transmission modes. For a wireless mode, multiple electrical conductive surfaces will be connected to various ECG lead wires present on the enclosure, connecting them to the multi-lead ECG AFE circuit. These ECG lead wires will be connected to other normal ECG electrodes, creating the wireless ECG signal acquisition system. In a wired mode, the enclosure will be connected to the power lines of the PCB, along with the multi-lead ECG AFE circuit.

In an embodiment, an accelerometer, a gyroscope and magnetometer will be placed in the topside of the PCB, parallel to the skin surface to accurately detect the angle of inclination along 3 axises to monitor patient bed position, head up elevation angle and respiratory rate. A power management and transmission circuit will be placed on the periphery of the PCB and away from the temperature sensor, AFE circuit to reduce the noises and distortions induced by them. A coin-cell holder will be present in the specially designed enclosure when the smart ECG electrode is set in wireless mode.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

This invention is illustrated in the accompanying drawings, throughout which like reference letters indicate corresponding parts in the various figures. The embodiments herein will be better understood from the following description with reference to the drawings, in which:

FIG. 1 is an illustration of a passive leg raising test that is commonly carried out in critical care units to identify patients who will benefit from further fluid therapy, according to embodiments as disclosed herein;

FIG. 2 is an illustration of a proposed system containing smart ECG electrode connected to hospital cloud system through a real-time patient monitoring system or a gateway through a wired or wireless medium, according to embodiments as disclosed herein;

FIG. 3 is an illustration of a smart ECG electrode which can act as an interface to pick up ECG signal and as well measure patient position, activity, head up elevation angle, skin temperature, and respiratory rate, patient heart rate, patient bed exit, arrhythmia, ischemic episodes, cardio-respiratory arrests and respiratory rate, according to embodiments as disclosed herein;

FIG. 4 is an illustration of continuous monitoring of head up elevation angle for patients who require VAP bundle compliance, according to embodiments as disclosed herein;

FIG. 5 is a block diagram of the patient monitoring system, according to an embodiment as disclosed herein;

FIG. 6 is a schematic view of system in which the patient monitoring system is connected with the cloud connected system and/or an electronic device, according to an embodiment as disclosed herein; and

FIG. 7 is a block diagram of the ECG electrode, according to an embodiment as disclosed herein.

### DETAILED DESCRIPTION OF INVENTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. Also, the various embodiments described herein are not necessarily mutually exclusive, as some embodiments can be combined with one or more other embodiments to form new embodiments. The term “or” as used herein, refers to a non-exclusive or unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

The present disclosure describes a patient monitoring system. The patient monitoring system includes an ECG electrode, comprises multiple built-in sensors, configured to measure patient information and stream the patient information. The patient information can be, for example, but not limited to the patient bed position, the patient head up elevation angle, the patient skin temperature, the patient respiratory rate, the patient heart rate, the patient bed exit, the arrhythmia, the ischemic episodes, and the cardio-respiratory arrests. Further, the patient monitor is configured to receive the streamed patient information from the ECG electrode and display the streamed patient information.

The present disclosure relates to the technical field of wearable device to solve unmet clinical needs by using a smart ECG electrode which like any normal ECG electrode can act as an interface to pick up ECG signal and as well measure patient position, activity, head up elevation angle, skin temperature, heart rate and respiratory rate.

The present disclosure describes a system and method of an ECG electrode with highly integrated circuitry which measures patient bed position, patient head up elevation angle, patient skin temperature measurement, patient respiratory rate, patient heart rate, patient falls or bed exits. The smart ECG electrode can early detect the sepsis, and recognize a cardiorespiratory arrest. Minimization of the form factor of the circuitry and the power source to fit into the existing ECG electrode form factor. Algorithm development for monitoring bed exits, patient activity classifier, step count etc. Custom ECG probe and ECG lead design for simultaneous transfer of power to the smart ECG electrode and as well acquire ECG signal, patient position and head up elevation angle. The respiratory rate is computed using the built-in sensors (ECG acquisition AFE, Accelerometer etc.) present in the smart ECG electrode. The skin temperature is monitored in real time to detect hyperthermia or hypothermia condition, essential for early detection of sepsis.

The present disclosure solves the above problems by system and method for managing patients with pressure ulcers, preventing VAP and sepsis by using a smart ECG electrode that can connect to other ECG electrodes forming a multi-lead ECG acquisition system in absence of a connection to multimodal monitors. It can stream ECG signal through wireless communication, and with a built-in processing unit, it can detect a wide range of arrhythmia, ischemic episodes, and cardio-respiratory arrests. It can also be used to measure heart rate, skin temperature and respiratory rate as part of an early warning system.

Information from the smart ECG electrode is relayed either through a wired or wireless medium and displayed through the patient monitor or using a gateway device. The patient monitoring system can then alert the nurses at regular intervals when the patient is due for a turn, thereby helping improving hospital turn protocol. The protocol is one of the major intervention in reducing the risk of pressure ulcers occurrences. It can also continuously monitor the head up elevation angle which is essential for ventilator-associated pneumonia (VAP) bundle compliance for reducing VAP occurrence.

With built-in processing unit in the smart electrode, it can detect and alert various conditions including various types of arrhythmias, bradycardia, tachycardia and cardiac or respiratory arrest enabling early detection and prompt response by resuscitation teams. With two such electrodes one on the chest and one on the lower limb it will be possible to aid clinicians in executing the passive leg raising test (PLR) correctly.

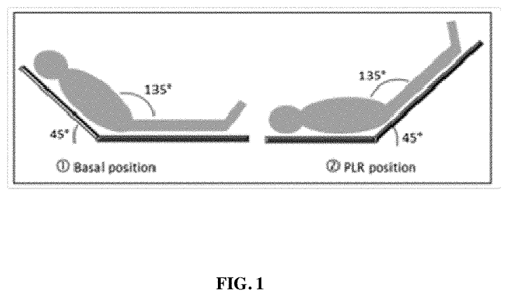

FIG. 1 is an illustration of a passive leg raising (PLR) test that is commonly carried out in critical care units to identify patients who will benefit from further fluid therapy, according to embodiments as disclosed herein.

In an embodiment in FIG. 1, passive leg raising test is a test that is commonly carried out in critical care units to identify patients who will benefit from further fluid therapy. The test involves placing the patient in a certain position before the test, basal position where the patient head up angle of 45 degrees and legs flat and then during the test in the PLR position, where the head and torso become recumbent and the legs are raised to a 45-degree angle. Both basal and PLR position is depicted in the FIG. 1. It is important to accurately measure these angles to improve the validity of the results and interpret them correctly. Presence of two smart electrodes one on the chest and the other on the leg will act as an invaluable guide in ensuring that the test is carried out in the manner prescribed and gives greater confidence in interpreting the results.

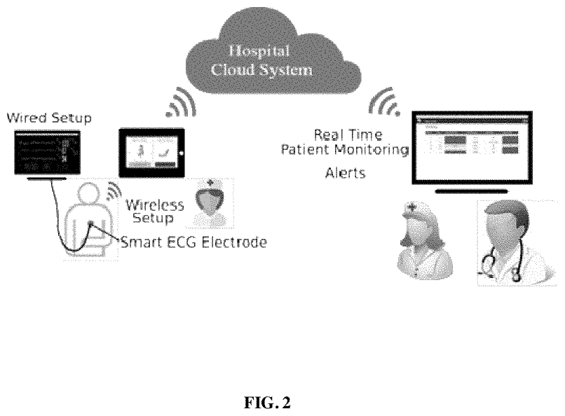

FIG. 2 is an illustration of a proposed system containing smart ECG electrode connected to hospital cloud system through a wired or wireless medium and real-time patient monitoring system, according to embodiments as disclosed herein.

In an embodiment in FIG. 2, the proposed system consists mainly of a smart ECG electrode which like any normal ECG electrode can act as an interface to pick up ECG signal and as well measure patient position, activity, head up elevation angle, skin temperature and respiratory rate. It updates the sensor information either using wired or wireless medium. When the electrode is connected using a special ECG probe, it can relay the information digitally. Data is then decoded and displayed using the standard patient monitor or a dedicated gateway device. If the wire is disconnected, it can also transmit wirelessly to the gateway device or patient monitor to display the multi-sensor information. In wireless communication, a provision is made for multiple smart ECG electrodes to communicate with a single gateway device. Data is pushed to the hospital cloud system using the gateway device.

In an embodiment in FIG. 2, the system alerts the caregiver at regular intervals according to various protocols like hospital turn protocol, VAP bundle etc. With continuous monitoring of patient position and activity, the system can alert the nurses when the patient is due for a turn, helping them comply with the hospital turn protocol. With the increase in compliance the risk of acquiring pressure ulcers reduces. Likewise, continuous monitoring of head up elevation angle in ventilated patients helps in maintaining the minimum set threshold. Nurses are alerted if the set standards are not met, thereby assisting them in complying with the VAP bundle which helps reduces the chances of acquiring ventilator-associated pneumonia (VAP). Using two electrodes one on the chest and the other on a leg will give an accurate measure of head up angle and leg raise angle which will enable the clinician to carry out the passive leg raising test in the prescribed manner.

In an embodiment in FIG. 2, smart ECG electrode can also monitor skin temperature in real time to detect hyperthermia or hypothermia condition, essential for early detection of sepsis. Additionally, it can also detect patient falls or bed exits and raise alarm if necessary. Respiratory rate can also be computed using built-in sensors and alerts the nurses when any abnormalities occur. When disconnected from the patient monitor, smart ECG electrodes can be connected to multiple ECG electrodes using special ECG wired setup acting as a multi-lead ECG acquisition system, wirelessly transmitting ECG signal to a patient monitor or gateway device. The ECG wired setup consists of multiple ECG lead wires with each connecting the mother node or smart ECG electrode to corresponding normal ECG electrode forming a multi leaded system. Built-in processing unit analyses the ECG signal in real-time and computes parameters including heart rate, respiratory rate etc. It can also enable identification of various types of arrhythmias, bradycardia, tachycardia, cardiorespiratory arrest and provide alerts to appropriate teams.

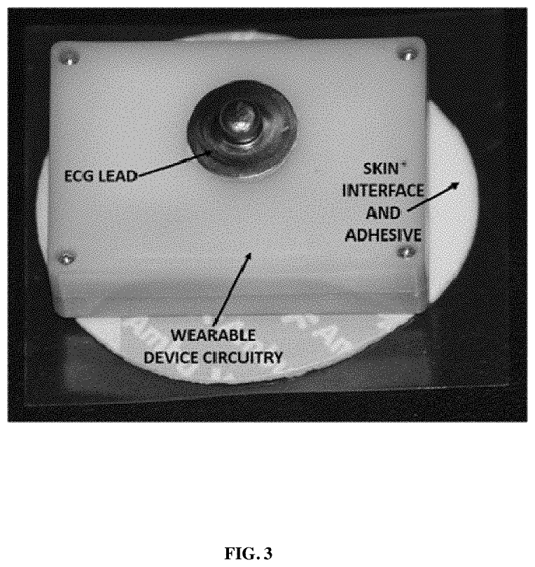

FIG. 3 is an illustration of a smart ECG electrode which can act as an interface to pick up ECG signal and as well measure patient position, activity, head up elevation angle, skin temperature and respiratory rate, according to embodiments as disclosed herein.

In an embodiment in FIG. 3, smart ECG electrode contains various sensors including a temperature sensor, an accelerometer, magnetometer, and a gyroscope. Additional circuitry for computation, ECG acquisition analog front end (AFE), power management, and transmission of data either in the wired or wireless medium is present. Smart ECG electrode will have the sensor, circuitry and the power source closely integrated and embedded on one side of the electrode, surrounding its metal lead. Smart ECG electrode can be powered directly through the patient monitor or gateway device using a special ECG probe. It can also be powered using a portable power source such as coin cell battery. FIG. 3 gives an estimation of how smart ECG electrode might look like. The height of the wearable device circuitry will be minimized The shape of the enclosed circuitry will not be rectangular in shape but approximately circular with a hole in the center accommodating the ECG lead. The adhesive and the skin interface will be similar to a normal ECG electrode.

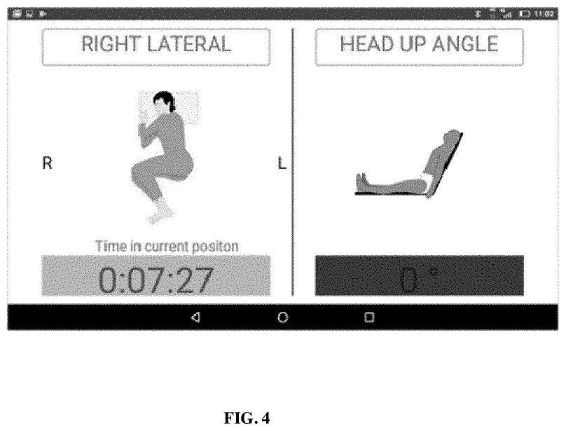

FIG. 4 is an illustration of continuous monitoring of head up elevation angle for patients who require VAP bundle compliance, according to embodiments as disclosed herein.

In an embodiment in FIG. 4, software design for the overall system can be divided into gateway interface software and hospital cloud system software. The gateway interface software runs on a tablet or cell phone or IOT node and provides an interface for the caregiver to interact with the wearable device. The gateway interface software displays a message asking the caregiver to attach the wearable device to the patient. Once it is attached, a gateway device or standard patient monitor connects to the smart ECG electrode either through wired or wireless means. It then starts continuous monitoring. The caregiver is then asked to set a threshold from a list of preset values for various parameters of the protocol. Interface once setup and running displays head up angle along with the patient's position and the time elapsed in the current position. Gateway device alerts the nurse through visual cues at regular intervals. When the patient is in compliance with the turn protocol, time in current position is shown in the FIG. 4. When a patient due for a turn in 15 minutes and if the patient needs to be turned immediately then it is shown in the FIG. 4. If nurses fail to comply with the turn protocol for more than one hour, head nurses are alerted through e-message once every half an hour until a valid turn is recognized. To account for sufficient decompression of tissues, the patient needs to stay in a new position for at least 15 minutes to be considered as a valid turn. Decompression algorithm present does the same ensuring the tissues are sufficiently depressurized.

In an embodiment in FIG. 4, a provision is made to enable continuous monitoring of head up elevation angle for patients who require VAP bundle compliance and head up elevation angle is continuously monitored displayed as shown in FIG. 4. The nurse is alerted visually if it's less than the set threshold. A representation of the same is shown in the FIG. 4. Gateway device will also be used to display the ECG signal streamed by the smart ECG electrode and acts as an interface to alert appropriate teams if any abnormalities are identified in the signal. Hospital cloud system software application running on the hospital's cloud system interacts with the gateway or tablet and provides centralized monitoring and continuous digital logging of the information transmitted by the smart ECG electrode. It also provides a centralized console to view and monitor all the patient details and desired parameters in real time connected through the gateway or tablet.

FIG. 5 is a block diagram of the patient monitoring system (100), according to an embodiment as disclosed herein. The monitoring system (100) includes the ECG electrode (110), a patient monitor (120), a memory (130), a processor (140) and a communicator (150). The arrangement and operations of the ECG electrode (110) are explained in conjunction with FIG. 7. The processor (140) is connected with the patient monitor (120), the memory (130), the processor (140) and the communicator (150).

The ECG electrode (110), comprises multiple built-in sensors (110e), configured to measure patient information and stream the patient information. The patient information can be, for example, but not limited to the patient bed position, the patient head up elevation angle, the patient skin temperature, the patient respiratory rate, the patient heart rate, the patient bed exit, the arrhythmia, the ischemic episodes, and the cardio-respiratory arrests. Further, the patient monitor (120) configured to receive the streamed patient information from the ECG electrode (110) and display the streamed patient information.

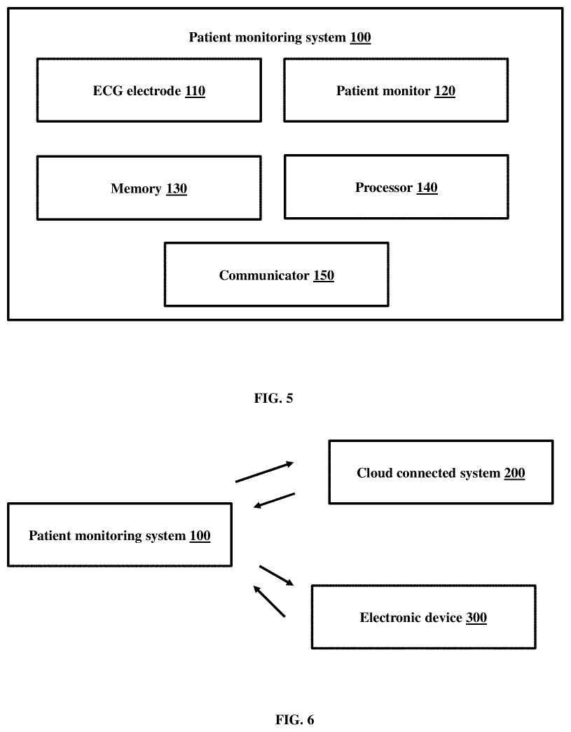

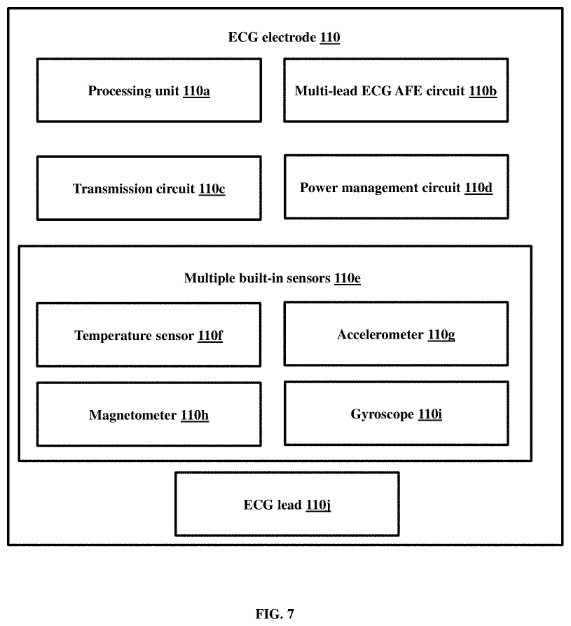

The streamed patient information is displayed through at least one of the gateway device and the cloud connected system (200) as shown in the FIG. 6.

In an embodiment, the patient monitor (120) alerts a caretaker at regular intervals based on at least one of a configurable hospital turn protocol and a configurable VAP bundle protocol. In an embodiment, the patient monitor (120) alerts a caretaker at regular intervals through the message, the alarm, the push notification or the like. In another embodiment, the patient monitor (120) alerts the caretaker at regular intervals based the predefined value. Further, the ECG electrode (110) is placed on the chest of the patient, and the multiple built-in sensors (110e) is placed on the lower limb of the patient to measure the head up angle of the patient and the leg raise angle of the patient to allow the caretaker to carry out the patient leg raising test.

Further, the processor (140) is configured to execute instructions stored in the memory (130) and to perform various processes. The communicator (150) is configured for communicating internally between internal hardware components and with external devices via one or more networks.

The memory (130) also stores instructions to be executed by the processor (140). The memory (130) may include non-volatile storage elements. Examples of such non-volatile storage elements may include magnetic hard discs, optical discs, floppy discs, flash memories, or forms of electrically programmable memories (EPROM) or electrically erasable and programmable (EEPROM) memories. In addition, the memory (130) may, in some examples, be considered a non-transitory storage medium. The term “non-transitory” may indicate that the storage medium is not embodied in a carrier wave or a propagated signal. However, the term “non-transitory” should not be interpreted that the memory (130) is non-movable. In some examples, the memory (130) can be configured to store larger amounts of information than the memory. In certain examples, a non-transitory storage medium may store data that can, over time, change (e.g., in Random Access Memory (RAM) or cache).

FIG. 7 is a block diagram of the ECG electrode (110), according to an embodiment as disclosed herein. The ECG electrode (110) includes a processing unit (110a), multi-lead ECG AFE circuit (110b), a transmission circuit (110c), a power management circuit (110d) and multiple built-in sensors (110e). The multiple built-in sensors (110e) includes a temperature sensor (110f), an accelerometer (110g), a magnetometer (110h) and a gyroscope (110i). In an embodiment, the ECG electrode (110) includes multiple built-in sensors (110e) and the processing unit (110a) that will be housed in a Printed Circuit Board (PCB) (not shown) which is of circular shape with a hole in a center of approximately the size of an ECG lead (110j). The smart ECG electrode has a physical structure and appearance of a normal clinical grade ECG electrode.

The PCB with the center hole will be attached to a set of passive ECG electrodes through the ECG lead (110j) and adhesive. Among various sensors, the temperature sensor (110f) will be attached on a backside of the PCB and closer to the skin to accurately measure the skin temperature. Possibly a punched hole of size of the temperature sensor (110f) might be present on the ECG electrode to place the sensor in close proximity with the skin to accurately detect the skin temperature. A single to multiple electrical conductive surface (not shown) will be present along the various regions of the PCB, connected to the specially designed enclosures based on various transmission modes. For a wireless mode, multiple electrical conductive surfaces will be connected to various ECG lead wires present on the enclosure, connecting them to the multi-lead ECG AFE circuit (110b). These ECG lead wires will be connected to other normal ECG electrodes, creating the wireless ECG signal acquisition system. In a wired mode, the enclosure will be connected to the power lines of the PCB, along with the multi-lead ECG AFE circuit (110b).

The accelerometer (110g), the gyroscope (110i) and the magnetometer (110h) will be placed in the topside of the PCB, parallel to the skin surface to accurately detect the angle of inclination along 3 axises to monitor patient bed position, head up elevation angle and respiratory rate. The power management circuit (110d) and the transmission circuit (110c) will be placed on the periphery of the PCB and away from the temperature sensor (110f) and the AFE, circuit (110b) to reduce the noises and distortions induced by them. A coin-cell holder will be present in the specially designed enclosure when the smart ECG electrode is set in wireless mode.

The embodiments disclosed herein can be implemented through at least one software program running on at least one hardware device and performing network management functions to control the elements.

The foregoing description of the specific embodiments will so fully reveal the general nature of the embodiments herein that others can, by applying current knowledge, readily modify and, or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

We claim:

1. A patient monitoring system (100), comprising:
an ECG electrode (110), comprising multiple built-in sensors (110e), configured to:
measure patient information pertaining to at least one of a patient bed position, patient head up elevation angle, patient skin temperature, patient respiratory rate, patient heart rate, patient bed exit, arrhythmia, ischemic episodes, and cardio-respiratory arrests, and
stream the patient information; and
a patient monitor (120) configured to:
receive the streamed patient information from the ECG electrode (110), and
display the streamed patient information,
wherein the ECG electrode (110) comprises a processing circuit (110a), a multi-lead ECG acquisition analog front end (AFE) circuit (110b), a power management circuit (110d), a transmission circuit (110c), and multiple built-in sensors (110e) having at least one of a temperature sensor (110f), an accelerometer (110g), a magnetometer (110h), and a gyroscope (110i);
wherein the ECG electrode (110) comprises the processing circuit (110a), the multi -lead ECG acquisition analog front end (AFE) circuit (110b), the power management circuit (110d), the transmission circuit (110c), and the multiple built-in sensors (110e) surrounded by an ECG lead (110j), and
wherein the ECG electrode (110) is placed on the chest of the patient, and wherein multiple built-in sensors (110e) is placed on a lower limb of the patient to measure a head up angle of the patient and a leg raise angle of the patient to allow a caretaker to carry out a patient leg raising test.

2. The patient monitoring system (100) claimed in claim 1, wherein the streamed patient information is displayed through at least one of a gateway device and a cloud connected system (200).

3. The patient monitoring system (100) as claimed in claim 1, wherein patient monitor (120) alerts a caretaker at regular intervals based on at least one of a configurable hospital turn protocol and a configurable ventilator-associated pneumonia (VAP) bundle protocol.

4. The patient monitoring system (100) as claimed in claim 1, wherein patient monitor (120) alerts a caretaker at regular intervals through a message.

5. The patient monitoring system (100) as claimed in claim 1, wherein patient monitor (120) alerts a caretaker at regular intervals based a predefined value.

6. The patient monitoring system (100) as claimed in claim 1, wherein the ECG electrode (110) acts as a master node that is connected to a set of passive ECG electrodes through a wired setup.

7. The patient monitoring system (100) as claimed in claim 1, wherein the patient monitor (110) is connected with an electronic device (300).

8. The patient monitoring system (100) as claimed in claim 1, wherein the multiple built-in sensors (110e) and the processing unit (110a) are housed in a Printed Circuit Board (PCB), wherein the PCB is provided with a centre hole in a center of approximately a size of the ECG lead (110j).

9. The patient monitoring system (100) as claimed in claim 10, wherein the PCB with the centre hole are attached to a set of passive ECG electrodes through the ECG lead (110j) using an adhesive.

10. The patient monitoring system (100) as claimed in claim 1, wherein the temperature sensor (110f) is attached on a backside of the PCB and closer to a skin of the patient to measure a skin temperature.

11. The patient monitoring system (100) as claimed in claim 1, wherein multiple electrical conductive surfaces are present along at least one region of the PCB, wherein the multiple electrical conductive surfaces are connected to enclosures based on a wireless mode and a wired mode.

12. The patient monitoring system (100 ) as claimed in claim 13, wherein multiple electrical conductive surfaces are connected to the ECG lead wires present on the enclosure in a wireless mode.

13. The patient monitoring system (100) as claimed in claim 13, wherein the enclosure is connected to power lines of the PCB along with the multi-lead ECG AFE circuit (110b) in a wired mode.

14. The patient monitoring system (100) as claimed in claim 1, wherein the accelerometer (110g), the gyroscope (110i) and the magnetometer (110h) are placed in a topside of the PCB, parallel to the skin surface of the patient, to detect the angle of inclination along 3 axis's to monitor patient bed position, head up elevation angle and respiratory rate.

15. The patient monitoring system (100) as claimed in claim 1, wherein the power management circuit (110d) and the transmission circuit (110c) are placed on a periphery of the PCB and away from the temperature sensor (100f) and the AFE circuit (111b). -)1
